(19) United States
(12) Patent Application Publication
(10) Pub. No.: US 2011/0224647 A1
(43) Pub. Date: Sep. 15, 2011

(54) BODY CAVITY DRAINAGE DEVICES AND RELATED METHODS

(76) Inventor: Harrison M. Lazarus, Salt Lake City, UT (US)

(21) Appl. No.: 13/045,274

(22) Filed: Mar. 10, 2011

Related U.S. Application Data
(60) Provisional application No. 61/312,878, filed on Mar. 11, 2010.

(51) Int. Cl. A61M 1/00; A61M 5/00

(52) U.S. Cl. 604/506; 604/540; 604/319

(57) ABSTRACT

Body cavity drainage devices and associated methods are disclosed herein. In some embodiments a body cavity drainage device comprises a drainage tube having a proximal end and a distal end; and an automated means to move the distal end of the drainage tube about a body cavity of a patient. In some embodiments, the distal end of the drainage tube may be moved by one or more of the application of a magnetic field, the insertion or withdrawal of fluid from a closed lumen, and manipulation by an external motion generator. In additional embodiments, a body cavity drainage device includes at least a second open lumen for the insertion of a fluid into a body cavity. In yet further embodiments, an internal tissue may be massaged from within a body cavity of a patient.

### CROSS-REFERENCE TO RELATED APPLICATIONS

This application claims the benefit of U.S. Provisional Application Ser. No. 61/312,878, filed Mar. 11, 2010, entitled “BODY CAVITY DRAINAGE DEVICE AND RELATED METHODS,” the disclosure of which is hereby incorporated herein by this reference in its entirety.

### BACKGROUND

1. Field

Embodiments of the invention relate to body cavity drainage devices and related methods.

2. State of the Art

Drainage devices, especially for the evacuation of a pleural cavity, may consist of a hollow flexible tube inserted through an incision into the pleural cavity. The shape and configuration of the pleural cavity often necessitates multiple incisions to be made to permit the drainage tube to reach various locations in the pleural cavity. The need for multiple incisions may generally result in an extended hospital stay from a patient suffering from a severe case of pleurisy.

### BRIEF SUMMARY

In some embodiments, a body cavity drainage device for a patient may comprise a drainage tube and an activation device. The drainage tube may have a proximal end and a distal end and a length sufficient to extend the distal end into the body cavity with the proximal end external to the patient. The activation device may be at least partially external to the patient for attachment to the proximal end of the drainage tube and adapted and structured to move the distal end of the drainage tube within a body cavity.

In additional embodiments, a body cavity drainage device may comprise a drainage tube having a proximal end and a distal end, and an automated means to move the distal end of the device about a body cavity of a patient.

In further embodiments, a body cavity drainage device may comprise a drainage tube that includes at least one open lumen having at least one opening at a proximal end and at least one opening at a distal end, and at least one closed lumen having at least one opening at a proximal end and a closed distal end.

In additional embodiments, a body cavity drainage device may comprise a drainage tube having a distal end sized and configured for insertion into a body cavity, a sleeve sized and configured to couple to a body cavity wall sealingly coupled with the drainage tube and slidable relative thereto, the distal end of the drainage tube positioned at a first side of the sleeve. The body cavity drainage device may further include a motion generator positioned on a second side of the sleeve and coupled to the drainage tube to effectuate movement of the distal end of the drain tube.

In further embodiments, a body cavity drainage device may comprise a drainage tube having a proximal end and a distal end, a first lumen and a second lumen. The first lumen may have at least one opening at the distal end and may be coupled to a suction source at the proximal end. The second lumen may have at least one opening at the distal end and may be coupled to a pressurized fluid source.

In yet further embodiments, methods of providing a treatment within a body cavity may comprise positioning a distal end of a drainage tube within the body cavity, and automatically moving the distal end of the drainage tube within the body cavity.

### BRIEF DESCRIPTION OF THE SEVERAL VIEWS OF THE DRAWINGS

FIG. 1 shows a detail view of a distal end portion of a drainage tube in a relaxed state, according to an embodiment of the present disclosure.

FIG. 2 shows a detail view of the distal end portion of the drainage tube shown in FIG. 1, wherein a closed lumen is filled with a pressurized fluid.

FIG. 3 shows a detail view of the distal end portion of the drainage tube shown in FIG. 1, wherein the fluid within the closed lumen is at an intermediate pressure.

FIG. 4 shows a cross sectional view of a drainage tube including a plurality of closed lumens, according to an additional embodiment of the present disclosure.

FIG. 5 shows a detail view of a distal end portion of the drainage tube of FIG. 4, wherein a closed lumen is filled with a pressurized fluid.

FIG. 6 shows a detail view of a distal end portion of the drainage tube of FIG. 4, wherein a different closed lumen is filled with a pressurized fluid.

FIG. 7 shows front view of a drainage tube including wires attached to the distal end of the drainage tube, according to an embodiment of the present disclosure.

FIG. 8 shows a cross-sectional view of the drainage tube of FIG. 7.

FIG. 9 shows a side view of the drainage tube of FIG. 7.

FIG. 10 shows front view of a drainage tube including wires attached to the distal end of the drainage tube and routed through lumens, according to an embodiment of the present disclosure.

FIG. 11 shows a cross-sectional view of the drainage tube of FIG. 10.

FIG. 12 shows a side view of the drainage tube of FIG. 10.

FIG. 13 shows a cross-sectional detail view of a distal end of a drainage tube including wires surrounding a portion of the distal end.

FIG. 14 shows a cross-sectional detail view of a distal end of a drainage tube including wires with stops thereon.

FIG. 15 shows a cross-sectional detail view of a distal end of a drainage tube including a single wire with a stop thereon.

FIG. 16 shows a schematic view of a drainage tube with wires, such as shown in FIGS. 7 and 10, in operation.

FIG. 17 shows a schematic view of the drainage tube of FIG. 16 moved to a second position.

FIG. 18 shows a schematic view of a device for operating the wires of a drainage tube with wires, such as shown in FIGS. 7 and 10, including a yoke.

FIG. 19 shows a schematic view of a device for operating the wires of a drainage tube with wires, such as shown in FIGS. 7 and 10, including separate reels.

FIG. 20 shows a schematic view of a device for operating the wires of a drainage tube with wires, such as shown in FIGS. 7 and 10, including linear actuators.

FIG. 21 shows a detail view of a distal end portion of a drainage tube including a magnetic end portion, according to an embodiment of the present invention.

FIG. 22 shows a schematic view of a magnetic field generator, according to an embodiment of the present invention.

FIG. 23 shows a schematic view of a linear motion generator in a fully extended state coupled to a drainage tube inserted into a body cavity, according to an embodiment of the present invention.

FIG. 24 shows a schematic view of the linear motion generator, drainage tube and body cavity of FIG. 23, wherein the linear motion generator is in a partially extended state.

FIG. 25 shows a schematic view of the linear motion generator, drainage tube and body cavity of FIG. 23, wherein the linear motion generator is in a refracted state.

FIG. 26 shows a schematic view of a circular motion generator in a first position coupled to a drainage tube inserted into a body cavity, according to an embodiment of the present invention.

FIG. 27 shows a schematic view of the circular motion generator, drainage tube and body cavity of FIG. 26, wherein the circular motion generator has been rotated one-quarter revolution.

FIG. 28 shows a schematic view of the circular motion generator, drainage tube and body cavity of FIG. 26, wherein the circular motion generator has been rotated one-half revolution.

FIG. 29 shows a schematic view of the circular motion generator, drainage tube and body cavity of FIG. 26, wherein the circular motion generator has been rotated three-quarters revolution.

FIG. 30 shows a detail view of a distal end portion of a drainage tube including a rotatable end portion, according to an embodiment of the present disclosure.

FIG. 31 shows a detail view of a distal end portion of a drainage tube including a separate open lumen, according to an embodiment of the present disclosure.

FIG. 32 shows a schematic detail view of a drainage tube having a distal end in a first position within a pleural cavity.

FIG. 33 shows a schematic detail view of the drainage tube of FIG. 32, wherein the distal end has been moved to a second position within the pleural cavity.

FIG. 34 shows a schematic detail view of a drainage tube having a distal end in a first position within a pleural cavity.

FIG. 35 shows a schematic detail view of the drainage tube of FIG. 34, wherein the distal end has been moved to a second position within the pleural cavity.

FIGS. 36 through 41 show schematic detail views of a drainage tube according to an embodiment of the present invention having a distal end positioned at different locations within a pleural cavity as a result of a movement of the distal end within the pleural cavity.

### DETAILED DESCRIPTION

In some embodiments, a drainage device may include a drainage tube configured to change shape in response to fluid pressure to facilitate the movement of a distal end thereof.

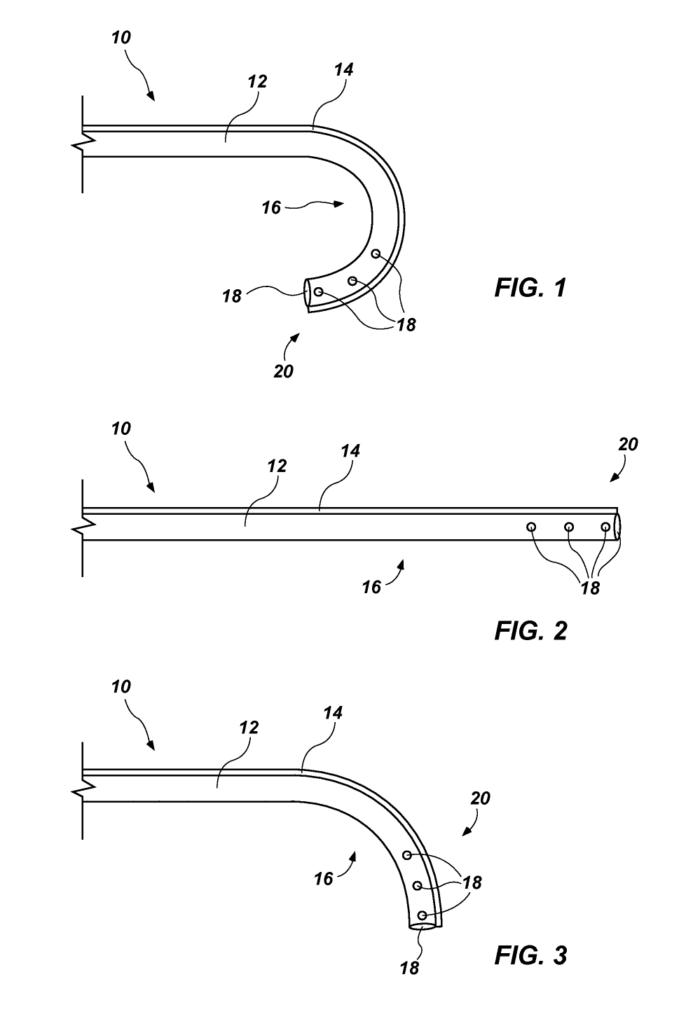

In one embodiment, as shown in FIG. 1, a drainage tube 10 may be generally configured as a catheter and may include a plurality of lumens. For example, the drainage tube 10 may include an open lumen 12 and a closed lumen 14. The drainage tube may further include a biasing structure 16, such as an elastic material region (e.g., flexible material region).

The open lumen 12 may include at least one opening 18 proximate to a first distal end 20 thereof and, in some embodiments, may include a plurality of openings 18 proximate to the first distal end 20. The one or more openings 18 may be defined by a one or more of apertures, porous regions, and other fluid permeable structures. The open lumen 12 may additionally include an opening at an opposing, second distal end that may be selectively coupled to a suction source, such as a vacuum (not shown).

The biasing structure 16 may be defined by an elastically deformable wall of the drainage tube 10. As a non-limiting example, the biasing structure 16 may bias the drainage tube 10 to extend along a generally arcuate path, such as shown in FIG. 1. In further embodiments, a biasing structure 16 may bias a drainage tube 10 to extend along a spiral path (not shown). In yet additional embodiments, a biasing structure 16 may bias a drainage tube 10 to extend along another path, such as a path having a generally linear shape.

The closed lumen 14 may extend longitudinally along at least a portion of the open lumen 12. In some embodiments, such as shown in FIG. 1, the closed lumen 14 may extend along the open lumen 12 and a closed, distal end of the closed lumen may be at least proximate to the distal end of the open lumen. The closed lumen 14 may additionally include an opening at an opposing, proximal end that may be selectively coupled to a fluid pressure source, such as a piston pump (e.g., a syringe) (not shown). When fluid within the interior of the closed lumen 14 is at a pressure at or near ambient pressure, the drainage tube 10 may be biased into a first path, such as a non-linear path (e.g., a generally arcuate path). However, as the fluid pressure within the closed lumen 14 is increased above the ambient pressure, such as by inflation with a gas (i.e., air) or insertion of a liquid (i.e., water), the closed lumen 14 may change in shape and cause the drainage tube 10 to extend along a different second path, such as a substantially linear path as shown in FIG. 2. In view of this, the shape of the drainage tube 10, and thus the position of a distal end 20 thereof, may be affected by varying the fluid pressure within the closed lumen 14. Conversely, when fluid within the interior of the closed lumen 14 is at a pressure at or near ambient pressure, the drainage tube 10 may be biased into a first path, such as a linear path. However, as the fluid pressure within the closed lumen 14 is increased above the ambient pressure, such as by inflation with a gas (i.e., air) or insertion of a liquid (i.e., water), the closed lumen 14 may change in shape and cause the drainage tube 10 to extend along a different second path, such as a substantially non-linear path (e.g., a generally arcuate path) as shown in FIG. 1. Additionally, by applying an intermediate fluid pressure within the closed lumen 14, the drainage tube 10 may assume an intermediate shape, such as shown in FIG. 3.

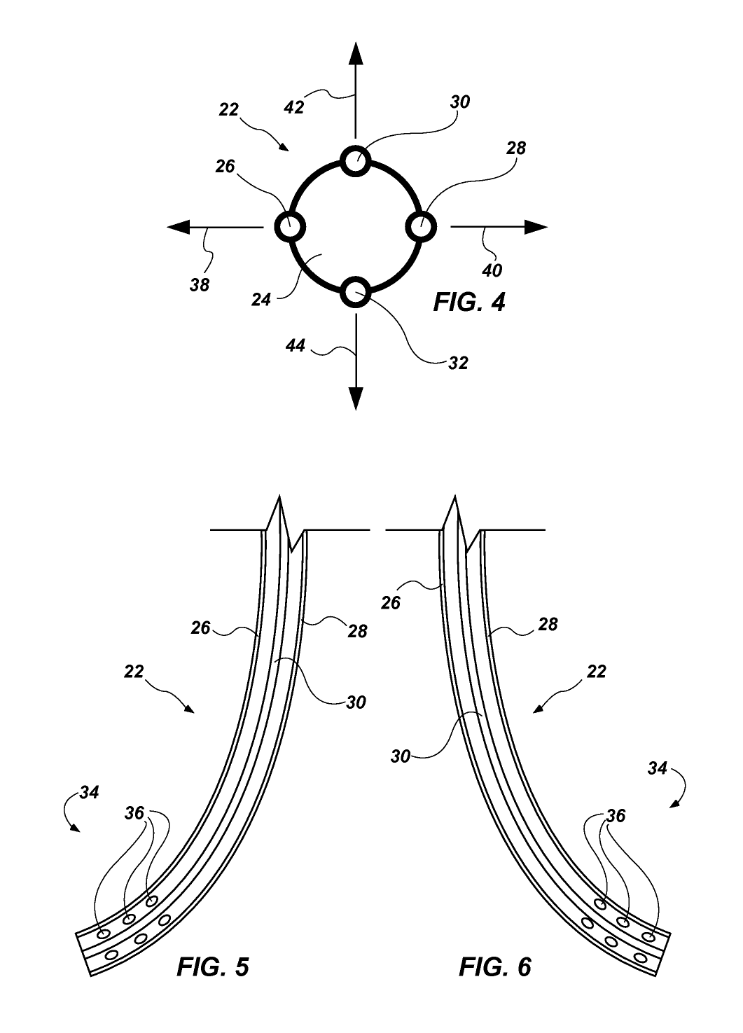

In additional embodiments, a drainage tube 22 may include an open lumen 24 for drainage and a plurality of closed lumens 26, 28, 30, 32 configured to facilitate the selective movement of a distal end in a number of directions in response to fluid pressure, as shown in cross-section in FIG. 4. For example, the drainage tube 22 may be biased along a generally linear path when the fluid within each closed lumen 26, 28, 30, 32 is at or near ambient pressure. When fluid pressure is applied to a first closed lumen 26 the first closed lumen 26 may change shape, such as a shape extending along a generally arcuate path, and facilitate the movement of the distal end 34 of the drainage tube 22, having openings 36 therein, in a first direction 38, as indicated in FIG. 4 and as shown in FIG. 5. When fluid pressure is applied to a second closed lumen 28 the second closed lumen 28 may change shape, such as a shape extending along another generally arcuate path, and facilitate the movement of the distal end 34 of the drainage tube 22 in a second direction 40, generally opposite the first direction 38, as indicated in FIG. 4 and as shown in FIG. 6. Similarly, when fluid pressure is applied to a third closed lumen 30 the third closed lumen 30 may change shape, such as a shape extending along yet another generally arcuate path, and facilitate the movement of the distal end of the drainage tube in a third direction 42, generally perpendicular to the first direction 38 and second direction 40, as indicated in FIG. 4. Finally, when fluid pressure is applied to a fourth closed lumen 32 the fourth closed lumen 32 may change shape and facilitate the movement of the distal end 34 of the drainage tube 22 in a fourth direction 44, generally opposite to the third direction 42, as indicated in FIG. 4.

In additional embodiments, an elongated structure may be utilized, rather than fluid pressure, to effectuate movement of a distal end of a drainage tube. For example, a wire or rod may be inserted into a closed lumen to change the shape thereof and facilitate movement of the distal end of the drainage tube.

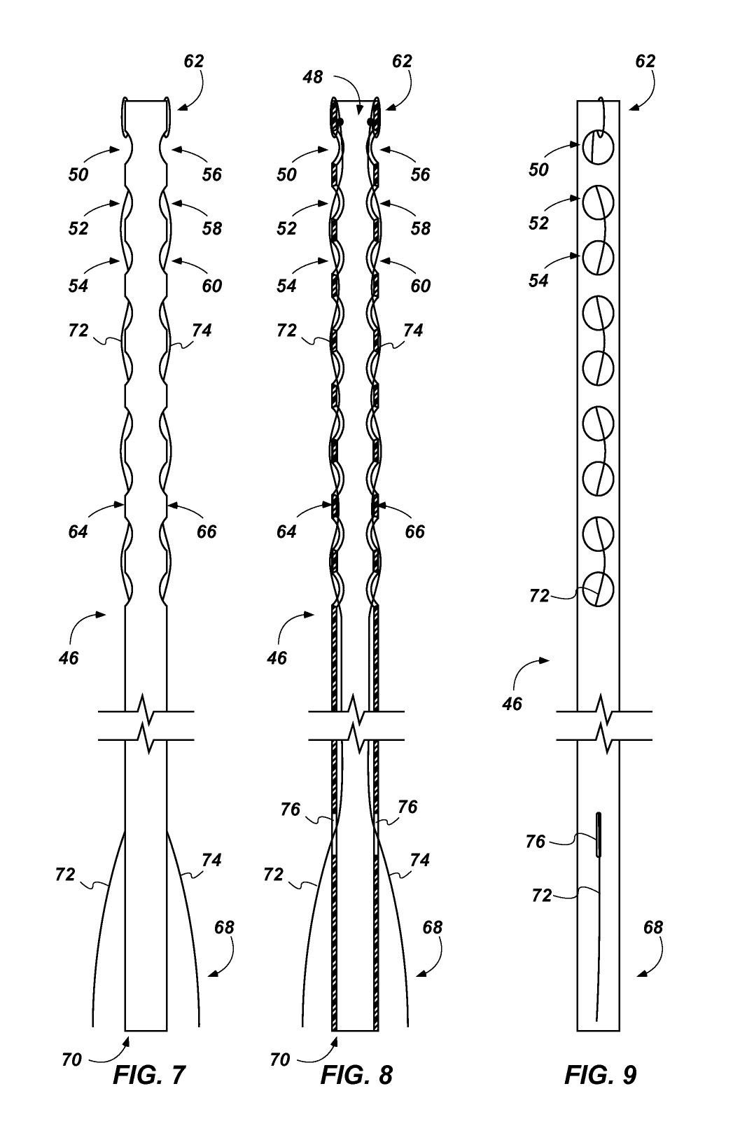

In some embodiments, at least one wire may be attached at or near a distal end of a drainage tube to effectuate the movement thereof, such as shown in FIGS. 7-15. As such embodiments may not utilize fluid pressure; such embodiments may not include any closed lumens. As used herein, the term “wire” is a broad term that encompasses any type of flexible elongated material capable of providing a pulling force and encompasses, by way of example and not limitation, metal wire, coated wire, polymer wire, woven or braided wire, string, yarn, line, cable, filament, lace, and cord.

In some embodiments, such as shown in FIGS. 7-9, a drainage tube 46 may be comprised of a single open lumen forming a central passage 48 (FIG. 8) through the drainage tube 46. The drainage tube 46 may be formed of a flexible material, such as a flexible polymer material, and may include a plurality of openings 50, 52, 54, 56, 58, 60 near a distal end 62. The drainage tube 46 may include a first plurality of openings 50, 52, 54 to the central passage 48 along a first side 64 of the drainage tube 46 near the distal end 62. The drainage tube 46 may also include a second plurality of openings 56, 58, 60 to the central passage 48 along an opposing, second side 66 of the drainage tube 46 near the distal end 62. A proximal end 68 of the drainage tube 46 may include an opening 70 configured for attachment to a vacuum source.

A first wire 72, such as a nickel titanium wire (e.g., a nitinol wire) having a polytetrafluoroethylene (e.g., TEFLON®) coating, may be attached to the drainage tube 46 at or near the distal end 62 of the drainage tube 46 and may be woven through the first plurality of openings 50, 52, 54. For example, the first wire 72 may extend from an outside surface of the drainage tube 46 and into the central passage 48 of the drainage tube 46 through a first opening 50 of the first plurality of openings 50, 52, 54. The first wire 72 may then extend along the central passage 48 for a distance and then extend to the outside of the drainage tube 46 through a second opening 52 of the first plurality of openings 50, 52, 54. The first wire 72 may then extend along the outside of the drainage tube 46 for another distance and extend back into the central passage 48 of the drainage tube 46 through a third opening 54 of the first plurality of openings 50, 52, 54. The first wire 72 may be woven in a similar manner through any number of additional openings formed along a first side 64 of the drainage tube 46. The first wire 72 may then extend through the central passage 48 of the drainage tube 46 toward the proximal end 68 of the drainage tube 46 and may extend out of the drainage tube 46 at or near the proximal end 68 of the drainage tube 46.

Similarly, an opposing, second wire 74, such as a nickel titanium wire (e.g., a nitinol wire) having a polytetrafluoroethylene (e.g., TEFLON®) coating, may be attached to the drainage tube 46 at or near the distal end 62 of the drainage tube 46 and may be woven through the second plurality of openings 56, 58, 60. For example, the second wire 74 may extend from an outside surface of the drainage tube 46 and into the central passage 48 of the drainage tube 46 through a first opening 56 of the second plurality of openings 56, 58, 60. The second wire 74 may then extend along the central passage 48 for a distance and then extend to the outside of the drainage tube 46 through a second opening 58 of the second plurality of openings 56, 58, 60. The second wire 74 may then extend along the outside of the drainage tube 46 for another distance and extend back into the central passage 48 of the drainage tube 46 through a third opening 60 of the second plurality of openings 56, 58, 60. The second wire 74 may be woven in a similar manner through any number of additional openings formed along a second side 66 of the drainage tube 46. The second wire 74 may then extend through the central passage 48 of the drainage tube 46 toward the proximal end 68 of the drainage tube 46 and may extend out of the drainage tube 46 at or near the proximal end 68 of the drainage tube 46.

As shown in FIGS. 7-9, the first and second wires 72, 74 may extend out of the drainage tube 46 through relatively small openings 76 near the distal end 68 of the drainage tube 46. Optionally, the first and second wires 72, 74 may extend out of the drainage tube 46 through openings 76 that provide a fluid seal around the first and second wires 72, 74. In view of this, there may be little or no leakage of ambient air into the drainage tube 46 through such openings 76 during operation thereof.

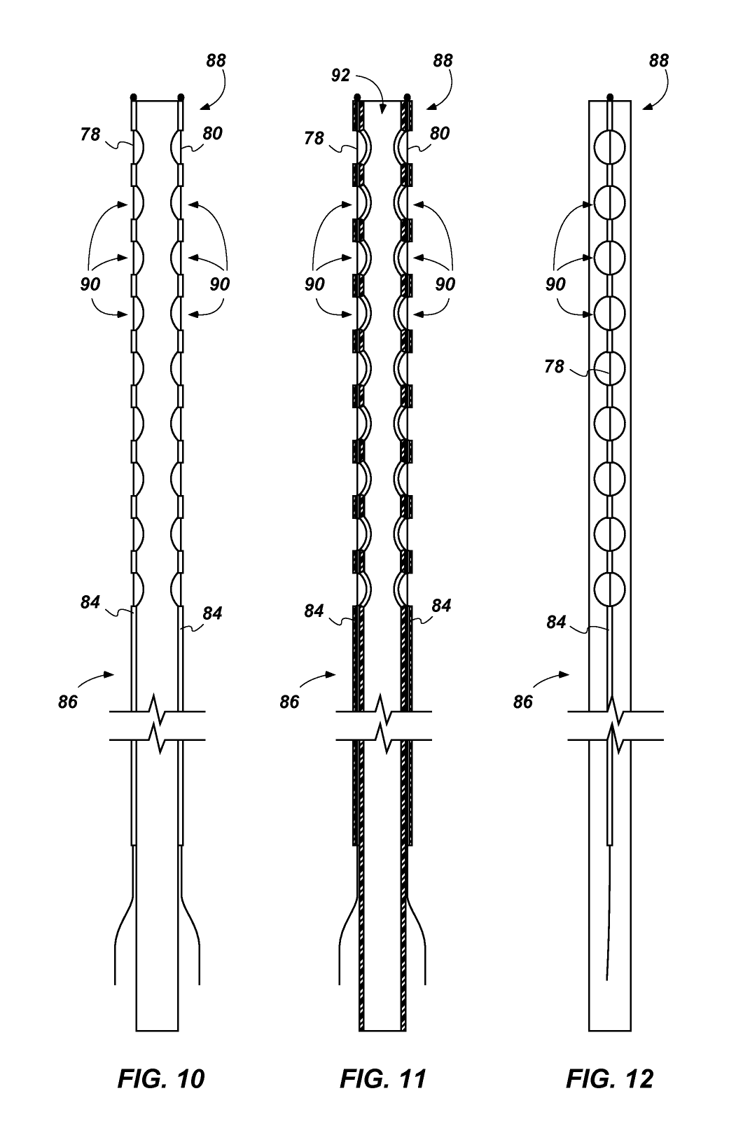

In additional embodiments, as shown in FIGS. 10-12, a first wire 78 and a second wire 80 may be inserted into relatively small lumens 82, 84 that are separate from a primary open lumen of a drainage tube 86 and the first and second wires 78, 80 may be attached to the distal end 88 of the drainage tube 86. In such embodiments, openings 90 into a central passage 92 of the drainage tube 86 may extend through the lumens 82, 84, creating a plurality of lumen 82, 84 segments. In view of this, the first and second wires 78, 80 may be exposed at the openings 90 into the central passage 92 of the drainage tube 86. As shown in FIGS. 10-12, the lumens 82, 84 and wires 78, 80 may be positioned on opposing sides of the drainage tube 86.

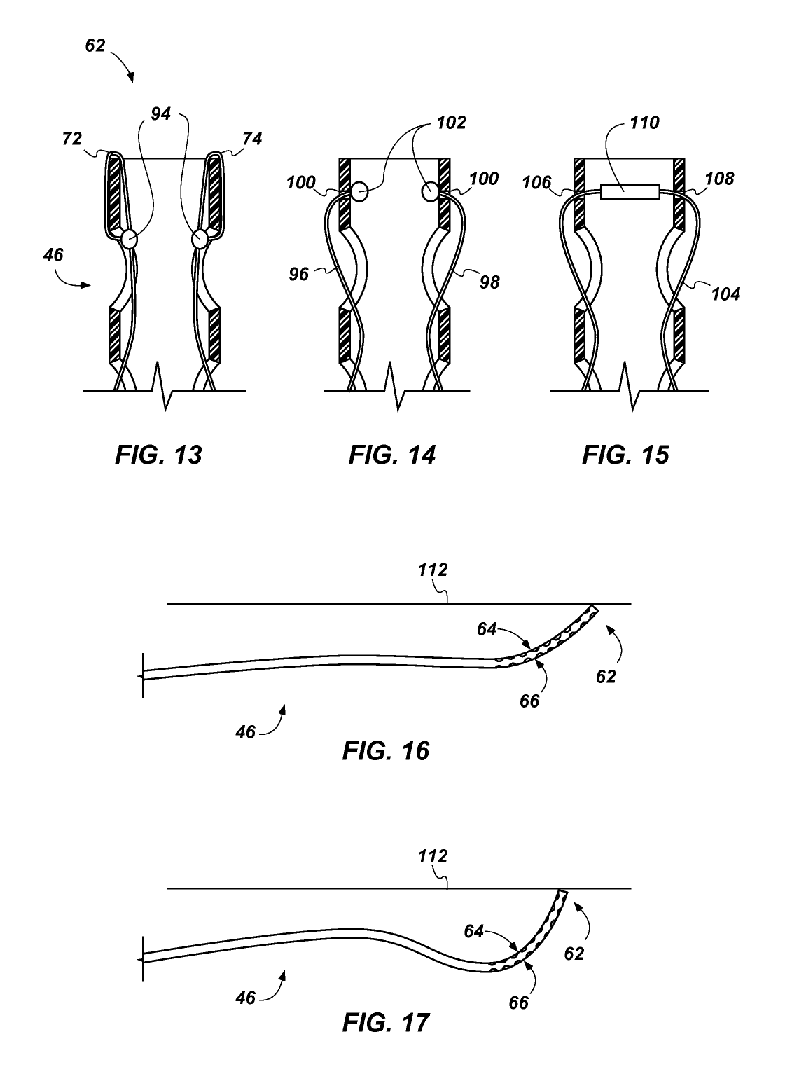

For embodiments that include wires, such as shown in FIGS. 7-12, the attachment of the wire 72, 74, 78, 80 to the drainage tube 46, 86 at or near the distal end 62, 88 may be accomplished by one of any number of attachment configurations. For example, each wire 72, 74 may wrap around a portion of the distal end 62 of the drainage tube 46, as shown in FIG. 13. Each wire 72, 74 may then be attached to itself at a joint 94, such as by a weld joint, a swaged or crimped joint, a knot, or another joint. For another example, a first wire 96 and a second wire 98 may extend through an opening 100 sized similar to a diameter of each respective wire 96, 98 and a stop 102, such as a weld bead or a swaged member, may be attached to a free end of each wire 96, 98 to prevent the free end of each wire 96, 98 from passing through the openings 100, respectively, such as shown in FIG. 14. In yet further embodiments, a single cable 104 may be utilized as a first cable and a second cable, such as shown in FIG. 15. When a single cable 104 is used the cable 104 may extend through first opening 106 and second opening 108 sized similar to a diameter of the wire 104 and a stop 110, such as a weld bead or a swaged member, may be attached to the cable 104 at a location between the first opening 106 and the second opening 108.

In operation, the distal ends 62, 88 of drainage tubes 46, 86 including wires 72, 74, 78, 80, such as shown in FIGS. 7-12, may be moved by the manipulation of the wires 72, 74, 78, 80 at the proximal end 68 of the drainage tube 46, 86. For example, as shown in FIGS. 16 and 17, and as described with further reference to features shown in FIGS. 7-10, when a tensile force is applied to the first wire 72 (e.g., when a pulling force is applied to the first wire 72 from the proximal end 68) the first side 64 of the drainage tube 46 may be put into compression. When the first side 64 of the drainage tube 46 is put into compression, the drainage tube 46 may flex at locations where the first plurality of openings 50, 52, 54 is located and the drainage tube 46 may bend in a first direction, as shown in FIGS. 16 and 17. Similarly, when a tensile force is applied to the second wire 74 (e.g., when a pulling force is applied to the second wire 74 from the proximal end 68) the second side 66 of the drainage tube 46 may be put into compression. When the opposing, second side 66 of the drainage tube 46 is put into compression the drainage tube may flex at locations where the second plurality of openings 56, 58, 60 is located and the drainage tube 46 may bend in an opposing, second direction (not shown). In view of this, the number of openings 50, 52, 54, 56, 58, 60 and the position of the openings 50, 52, 54, 56, 58, 60 in the drainage tube 46 may be chosen to cause the drainage tube 46 to exhibit a desired change in shape upon manipulation of the wires 72, 74 from the proximal end 68.

Additionally, when the distal end 62 of the drainage tube 46 contacts an obstruction 112, the distal end 62 of the drainage tube 46 may remain in contact with the edge of the obstruction 112 as the drainage tube 46 continues to flex, as shown in FIGS. 16 and 17. This may allow the edges of a body cavity, or the edges of an object within the body cavity, to be efficiently cleared of fluids and improve the drainage of the body cavity.

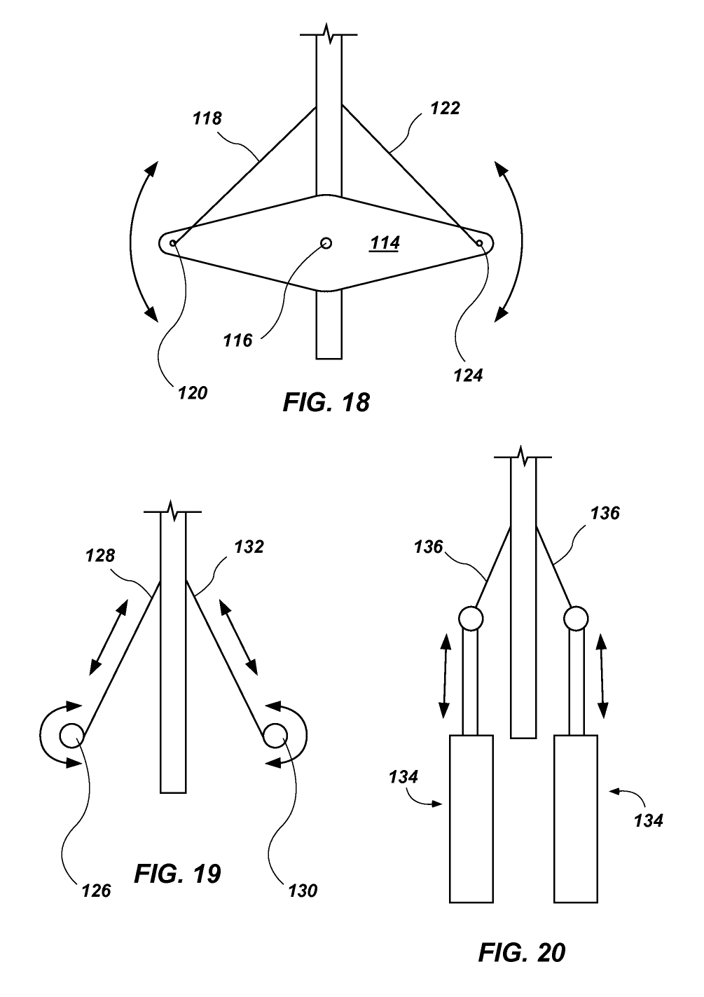

For drainage tubes including wires, such as shown in FIGS. 7-12, any number of mechanisms may be attached to the proximal ends of the wires to selectively apply tensile force to the wires. In some embodiments, the proximal ends of the wires may be manipulated directly by a physician. In further embodiments, the wires may be manipulated automatically by a programmed device. For example, in some embodiments a motor (not shown) may be attached to a yoke 114 at a central location 116 thereof, such as shown in FIG. 18. When the motor is turned in a first direction, a first wire 118 may be pulled by the yoke 114 and a tensile force may be applied to the first wire 118, which may be attached at a first end 120 of the yoke 114. Similarly, when the motor is turned in an opposing, second direction, a second wire 122 may be pulled by the yoke 114 and a tensile force may be applied to the second wire 122, which may be attached to a second end 124 of the yoke 114. In additional embodiments, a first reel 126 may be attached to a first wire 128 and a second reel 130 may be attached to a second wire 132, and motors (not shown) attached to each reel 126, 130 and may selectively apply a tensile force to each wire 128, 132, respectively, such as shown in FIG. 19. In yet further embodiments, a linear actuator 134, such as a pneumatic, hydraulic, or electric linear actuator, may be attached to each wire 136 and may selectively apply a tensile force to each wire 136, respectively, such as shown in FIG. 20.

Although the embodiments shown in FIGS. 7-20 include a first wire and a second wire, different numbers of wires may be utilized. For example, a drainage tube may include only a first wire and not include a second wire. Additionally, a drainage tube may include a first wire, a second wire, and any number of additional wires. Generally, the more wires that are included, the greater the range of motion that may be achieved with a distal end of a drainage tube. However, two opposing wires may provide sufficient range of motion for many therapeutic uses, such as for draining fluid from a body cavity, such as a pleural cavity.

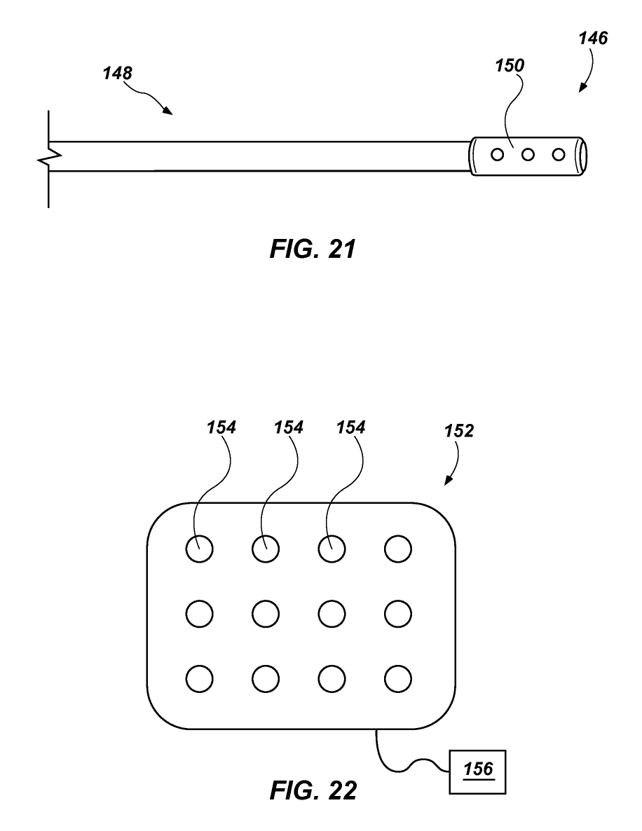

In additional embodiments, such as shown in FIG. 21, movement of a distal end 146 of a drainage tube 148 may be facilitated by an applied magnetic force, which may be generated outside of a patient's body. In such embodiments, the drainage tube 148 may include a magnetic material portion (i.e., a material that may experience a force in response to an applied magnetic field), such as a ferromagnetic material portion (e.g., iron, nickel, cobalt, gadolinium, neodymium, samarium and alloys thereof). For example, the drainage tube 148 may include a magnetic structure 150 formed of a magnetic material positioned near the distal end 146 thereof, such as shown in FIG. 16. The magnetic structure 150 may be formed of a material that is different than a majority of the drainage tube 148, or may be defined by a region doped with a magnetic material. In view of this, a magnetic force may be applied to the distal end 146 of the drainage tube 148 by the application of a magnetic field generating device 152, such as shown in FIG. 22, which may cause the distal end 146 of the drainage tube 148 to move in response thereto.

In some embodiments, the magnetic field generating device 152 may be configured as an external covering that may be worn by a patient (e.g., as a vest) or may be draped over the patient. The magnetic field generating device 152 may deliver a reconfigurable magnetic field that may cause the distal end 146 of a drainage tube 148 positioned within a body cavity, such as a pleural cavity of a patient, to move about the body cavity. For example, the magnetic field generating device 152 may include a plurality of temporary magnets 154 (e.g., electromagnets), distributed thereabout that may be selectively activated to generate a number of magnetic field configurations to affect the movement of the distal end 146 of the drainage tube 148 to a number of positions within the body cavity. In additional embodiments, a magnetic field generating device may include permanent magnets that may be utilized to generate a number of magnetic field configurations. For example, a permanent magnet may be moved about or the orientation of a plurality of permanent magnets relative to one another may be manipulated to generate different magnetic field configurations. Additionally, the magnetic field generation may be automatically activated and manipulated, such as by a programmed control module 156.

In additional embodiments, motion generators positioned external to a patient may facilitate movement of a distal end of a drain tube positioned within a body cavity of the patient.

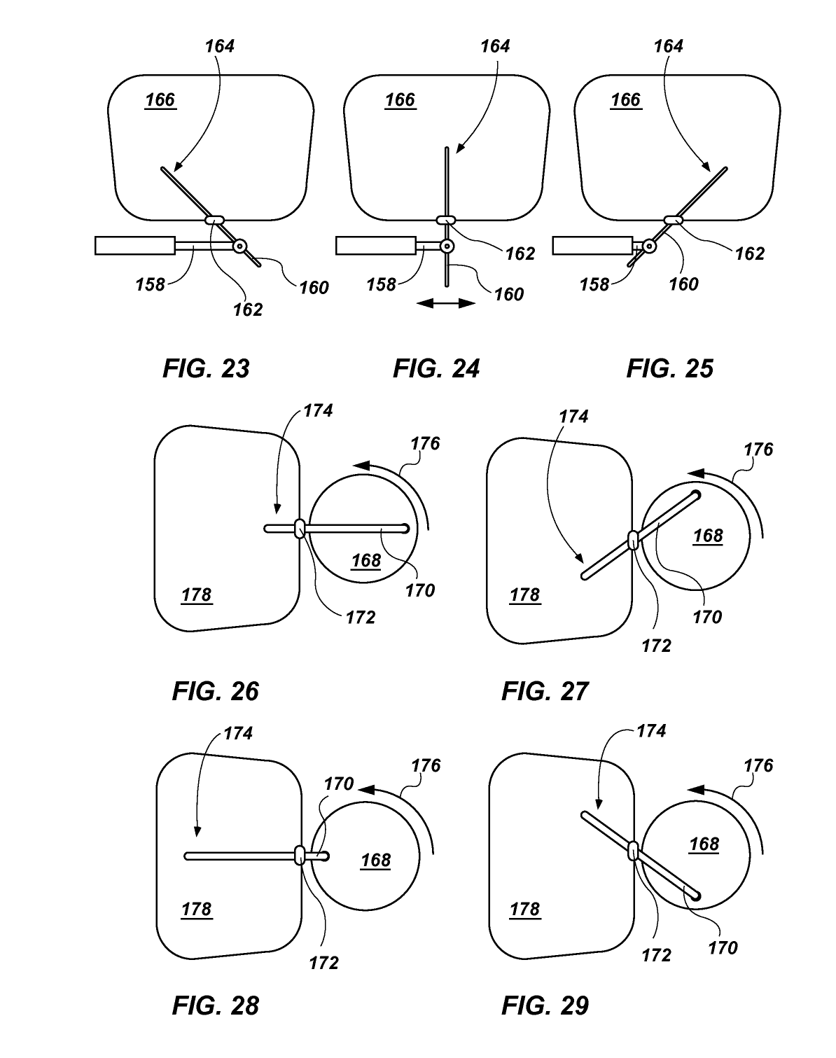

In some embodiments, a linear motion generator, such as a piston 158, may be attached to a drainage tube 160, such as shown in FIGS. 23 through 25. In such embodiments, the drainage tube 160 may be positioned within a sleeve 162 that may be positioned within a cavity wall, such as a chest wall, of a patient. A distal end 164 of the drainage tube 160 may be positioned within a body cavity 166 at a first side of the sleeve 162 and the piston 158 may be coupled to the drainage tube 160 at an opposing, second side of the sleeve 162. The sleeve 162 may be configured to allow the movement of the drainage tube 160 relative to the sleeve 162, and thus relative to the patient's cavity wall as the sleeve 162 may be configured to contact and be coupled to the patient's cavity wall, while maintaining a seal between the sleeve 162 and the drainage tube 160. In view of this, in some embodiments, the distal end 164 of the drainage tube 160 may be inserted into a body cavity of a patient, such as a pleural cavity, and an outer wall of the sleeve 162 may be coupled to the chest wall of the patient. Upon insertion, the piston 158 may be extended and/or refracted to facilitate movement of the distal end 164 of the drainage tube 160 within the patient's body cavity 166, as shown in FIGS. 23 through 25.

In additional embodiments, a circular motion generator, such as a wheel 168 attached to a motor, may be attached to a drainage tube 170, such as shown in FIGS. 26 through 29. In such embodiments, the drainage tube 170 may be positioned within a sleeve 172 that may be positioned within a cavity wall, such as a chest wall, of a patient. A distal end 174 of the drainage tube 170 may be positioned at a first side of the sleeve 172 and the wheel 168 may be coupled to the drainage tube 170 at an opposing, second side of the sleeve 172. The sleeve 172 may be configured to allow the movement of the drainage tube 170 relative to the sleeve 172, and thus relative to the patient's cavity wall as the sleeve 172 may be configured to contact and be coupled to the patient's cavity wall, while maintaining a seal between the sleeve 172 and the drainage tube 170. In view of this, in some embodiments, the distal end 174 of the drainage tube 170 may be inserted into a body cavity of a patient, such as a pleural cavity, and an outer wall of the sleeve 172 may be coupled to the chest wall of the patient. Upon insertion, the wheel 168 may be selectively rotated, such as in a direction indicated by the arrow 176, to facilitate movement of the distal end 174 of the drainage tube 170 within a patient's body cavity 178, as shown in FIGS. 26 through 29.

In embodiments utilizing motion generators external to a patient, or other embodiments wherein a portion of a drainage tube may be inserted and/or refracted from the patient outside of a sterile environment, at least a portion of the drainage tube may be covered by a flexible covering that may be attached to the sleeve, such as a flexible plastic film (not shown).

Although linear and circular motion generators have been shown and described in particular embodiments herein, other motion generators, as will be recognized by a person of ordinary skill in the art, may also be utilized to generate other simple motions or compound motions.

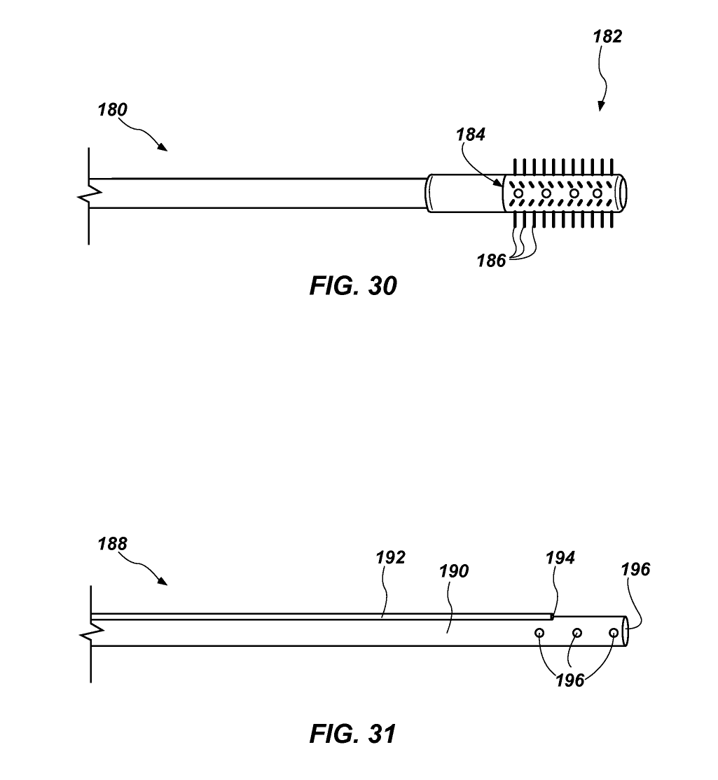

In additional embodiments, a drainage tube 180 may include a locomotion device positioned at least proximate to a distal end 182 thereof. For example, the drainage tube may include a distal end portion 184 that is rotatable, as shown in FIG. 30. Optionally, the rotatable end portion 184 may further include protrusions 186 extending from a surface thereof, such as similar to relatively soft bristles of a brush. In view of this, upon the selectable rotation of the rotatable end portion 184, the distal end 182 of the drainage tube 180 may move about a body cavity, such as a pleural cavity. Additionally, the protrusions 186 may be utilized to massage internal body tissue, such as lung tissue. The locomotion device may be powered by electric power, hydraulic power, or another power source. As non-limiting examples, power may be provided by directing electricity through a wire positioned within the drainage tube 180 or by directing fluid through lumens of the drainage tube 180, such as by fluid injection or by fluid suction. Although a rotatable end portion 184 is shown and described with reference to FIG. 30, other locomotion devices may be positioned at least proximate to a distal end 182 of the drainage tube 180 to provide locomotion of the distal end 182 of the drainage tube 180 relative to a patient's body cavity. For example, any number of mechanical device configurations (e.g., a microelecromechanical system (MEMS)) may be utilized to provide mechanical locomotion, such as including rotatable wheels, articulated legs, and other mechanisms.

In some embodiments, a drainage tube 188 may include a plurality of open lumens 190, 192, such as shown in FIG. 31. In such embodiments, each open lumen 190, 192 may extend along a length of the drainage tube 188 and have at least one opening 194, 196 at a distal end thereof. A first open lumen 190 may have another opening at an opposing, proximal end that may be coupled to a suction source (not shown). A second lumen 192 may also have an opening at an opposing, proximal end (not shown). However, the opening at the proximal end of the second lumen 192 may be coupled to a fluid source, rather than a suction source. In view of this, a fluid may be injected into a body cavity from the fluid source through the opening 194 of the second open lumen 192 while fluid is removed from the body cavity through the openings 196 of the first open lumen 190. For example, fluids such as one or more of antibiotics, saline water, enzymes, and other fluids may be injected through the opening 194 of the second open lumen 192 into a body cavity such as for one or more of irrigation, blood thinning, medication delivery, tissue stimulation, and other treatments. Simultaneously, fluid may be removed from the body cavity through the openings 196 of the first open lumen 190.

In some embodiments, methods of utilizing a drainage tube having a moveable distal end may include determining a size and shape of a body cavity, preselecting a range of movement for a drainage tube, inserting the drainage tube into the body cavity and moving the distal end of the drainage tube within the body cavity according to the preselected range of movement. For example, body imaging technology such as x-ray imaging, ultrasound imaging, magnetic resonance imaging (MRI), and computed tomography (CT) scanning may be utilized to determine the size and shape of a body cavity. Additionally, a size and shape of a body cavity may be estimated by external measurements of a patient without utilizing body imaging technology. Next, a range of movement may be selected by utilizing the estimated size and shape of the body cavity and specific treatment objectives. For example, a range of movement about a curved surface, such as a shape of a surface of a pleural cavity defined by a chest wall, may be selected for the cleaning and drainage of the pleural cavity. After the range of movement is selected, a drainage tube may be configured to achieve the selected range of movement and may be inserted into the body cavity. After insertion into the body cavity, the distal end of the drainage tube may be moved within the body cavity according to the preselected range of movement to facilitate a specific treatment.

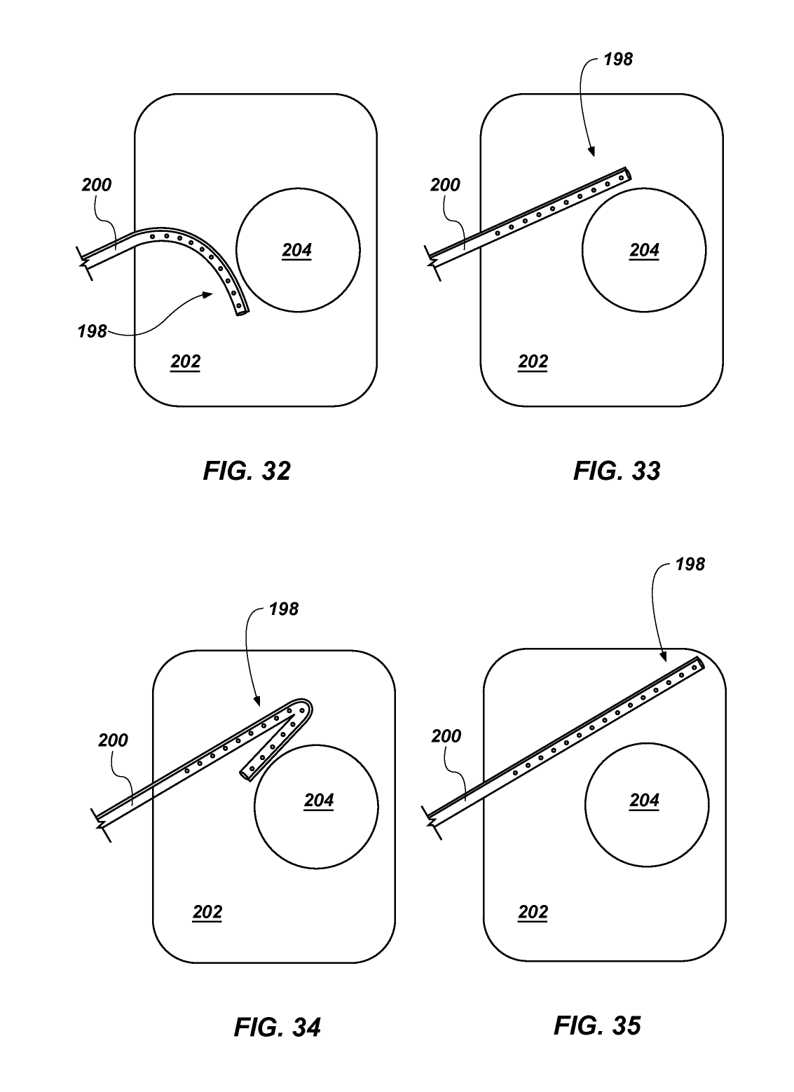

In view of the foregoing, embodiments may be utilized to treat and drain body cavities of patients. For example, embodiments may be utilized to move a distal end 198 of a drainage tube 200 within a pleural cavity 202, providing drainage about a lung 204, as shown in FIGS. 32 and 33 and FIGS. 34 and 350, to treat and drain an empyema or other flowable liquid, gaseous or semisolid matter from the pleural cavity 202 or from another body cavity. Embodiments that include a drainage tube having a distal end that may be moveable after insertion into a body cavity may be utilized to remove substantially all of a flowable material from a body cavity. Additionally, the moveable distal end may provide other beneficial treatments, such as the massaging of tissue, such as lung tissue. Furthermore, embodiments may provide the infusion of medication, the introduction of irrigation fluids, enzymes or other treatments into specific regions of a body cavity or may distribute such treatments over substantially all of a body cavity.

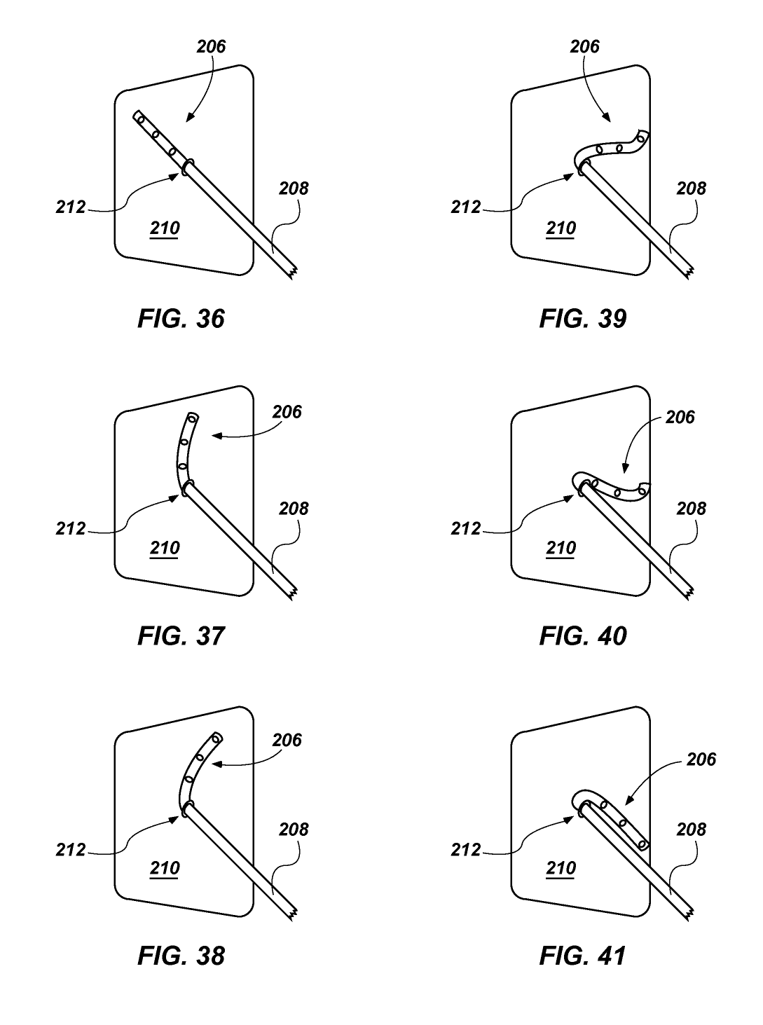

In some embodiments, a distal end 206 of a drainage tube 208 may move about a body cavity in a snake-like motion, as shown in FIGS. 36 through 41. For example, and as shown in FIGS. 36 through 41, the drainage tube 208 may be inserted at a generally central region of a pleural cavity 210 and the distal end 206 of the drainage tube 208 may move about an upper portion of the pleural cavity 210 from the insertion point 212, such as in a snake-like motion. Similarly, the drainage tube 208 may move about the lower portion of the pleural cavity 210 from the insertion point 212.

In view of the foregoing, access to various regions of a body cavity by a distal end of a drainage tube may be achieved at a single point of entry, and without requiring multiple incisions. Additionally, a drainage tube according to embodiments such as described herein may have a significantly smaller diameter than conventional drainage tubes, yet have superior efficacy. A relatively small diameter drainage tube may facilitate insertion, reduce pain and discomfort experienced by a patient, reduce bleeding, and decrease recurrent pneumothorax on withdrawal of the drainage tube. The device may be especially effective in draining or evacuating fluid from the complex configuration of a pleural cavity.

While the invention may be susceptible to various modifications and alternative forms, specific embodiments have been shown by way of example in the drawings and have been described in detail herein. However, it should be understood that the invention is not intended to be limited to the particular forms disclosed. Additionally, embodiments and features of the invention shown and/or described separately may be combined.

What is claimed is:

1. A body cavity drainage device for a patient comprising:
a drainage tube having a proximal end and a distal end and a length sufficient to extend the distal end into the body cavity with the proximal end external to the patient; and
an activation device at least partially external to the patient for attachment to the proximal end of the drainage tube adapted and structured to move the distal end of the drainage tube in the body cavity.

2. The body cavity drainage device of claim 1, wherein the drainage tube comprises a plurality of openings at the distal end and an opening at the proximal end coupled to a suction source.

3. A body cavity drainage device of claim 2, further comprising an automated means to move the distal end of the drainage tube about a cavity of a patient.

4. The body cavity drainage device of claim 3, further comprising at least one wire attached at or near the distal end of the drainage tube and extending to a proximal end of the drainage tube.

5. The body cavity drainage device of claim 4, further comprising a plurality of wires attached at or near the distal end of the drainage tube and extending to a proximal end of the drainage tube.

6. The body cavity drainage device of claim 4, wherein the at least one wire is woven through a plurality of openings in the drainage tube, extending along both an exterior of the drainage tube and an interior of the drainage tube.

7. The body cavity drainage device of claim 4, wherein the at least one wire extends through at least one segmented lumen.

8. The body cavity drainage device of claim 7, wherein the at least one wire is exposed at the location of at least one opening into a central passageway of the drainage tube.

9. The body cavity drainage device of claim 4, wherein the at least one wire is coupled to an automated actuation mechanism at the proximal end.

10. The body cavity drainage device of claim 9, wherein the automated actuation mechanism comprises a yoke attached to the at least one wire and a motor attached to the yoke.

11. The body cavity drainage device of claim 9, wherein the automated actuation mechanism comprises at least one reel attached to the at least one wire.

12. The body cavity drainage device of claim 9, wherein the automated actuation mechanism comprises at least one linear actuator attached to the at least one wire.

13. The body cavity drainage device of claim 2, wherein the drainage tube further comprises:
at least one open lumen having at least one opening at a proximal end and at least one opening at a distal end; and
at least one closed lumen having at least one opening at a proximal end and a closed distal end.

14. The body cavity drainage device of claim 13, further comprising:
a vacuum source coupled to the at least one opening at the proximal end of the at least one open lumen; and
a fluid pressure source coupled to the at least one opening at the proximal end of the at least one closed lumen.

15. The body cavity drainage device of claim 13, wherein a shape of the drainage tube and a position of the distal end of the at least one open lumen is dependent at least in part upon a fluid pressure within the at least one closed lumen.

16. The body cavity drainage device of claim 13, further comprising a plurality of closed lumens configured to facilitate the selective movement of the distal end of the drainage tube in a number of directions in response to fluid pressure.

17. The body cavity drainage device of claim 13, further comprising a wire or rod sized and configured for insertion into the closed lumen to change the shape thereof and effectuate movement of the distal end of the drainage tube.

18. A method of providing a treatment within a body cavity comprising:
positioning a distal end of a drainage tube within the body cavity; and
automatically moving the distal end of the drainage tube within the body cavity.

19. The method of claim 18, further comprising generating at least one magnetic field to move the distal end of the drainage tube within the body cavity.

20. The method of claim 18, further comprising massaging an internal body tissue with the distal end of the drainage tube.

21. The method of claim 18, further comprising injecting a fluid into the body cavity through a second lumen of the drainage tube.

22. The method of claim 18, further comprising:
determining a size and shape of the body cavity;
preselecting a range of movement for the drainage tube; and
wherein automatically moving the distal end of the drainage tube within the body cavity further comprises moving the distal end of the drainage tube within the body cavity according to the preselected range of movement.

23. The method of claim 22, wherein determining a size and shape of the body cavity comprises imaging the body cavity with body imaging technology.

24. The method of claim 22, wherein determining a size and shape of the body cavity comprises estimating a size and shape by external measurements of a patient.

25. The method of claim 22, wherein preselecting a range of movement for the drainage tube comprises selecting a range of movement that corresponds to the determined size and shape of the body cavity.

26. A body cavity fluid evacuation device, comprising:
an elongated tube comprising an open lumen and having a proximal end and a distal end, the distal end having at least one opening therein to the open lumen, the tube sized and adapted to be inserted through an incision in a patient and into a body cavity and a length to reach a remote region of the body cavity; and
an activation means positioned at least partially at the proximal end of the tube to controllably move the distal end of the elongated hollow tube within the body cavity.

27. The body cavity fluid evacuation device of claim 26, wherein the activation means comprises a wire connected at least proximate to the distal end of the tube, wherein at least a portion of the wire is disposed within the open lumen.
